(19) United States
(12) Patent Application Publication
(10) Pub. No.: US 2010/0209540 A1
(43) Pub. Date: Aug. 19, 2010

(54) INHALATION FORMULATION FOR TREATING AND PROPHYLACTIC USE IN BACTERIA, MYCOBACTERIAL AND FUNGAL RESPIRATORY INFECTIONS

(75) Inventors: W. Randolph WARNER, Punta Gorda, FL (US); Werner Gutmann, Powhatan, VA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: PharmaCaribe, Punta Gorda, FL (US)

(21) Appl. No.: 12/612,826

(22) Filed: Nov. 5, 2009

Related U.S. Application Data
(60) Provisional application No. 61/111,515, filed on Nov. 5, 2008.

(51) Int. Cl. A61K 33/14; A61P 11/00; A61P 31/12; A61P 31/04

(52) U.S. Cl. 424/680

(57) ABSTRACT

A composition for the treatment and prophylactic management of bacterial and fungal respiratory infections is provided. The composition may contain a combination of a quaternary ammonium compound and hypertonic saline to be administered to a patient in need thereof.

### CROSS-REFERENCES TO RELATED APPLICATIONS

This application claims priority to and the benefit of 35 U.S.C. §119(e) to provisional application Ser. No. 61/111,515, filed Nov. 5, 2008, the disclosure of which is expressly incorporated by reference herein in its entirety.

### BACKGROUND OF THE INVENTION

1. Field of the Invention

The invention generally relates to compositions and methods of administering a composition containing a cationic quaternary ammonium compound in combination with a hypertonic saline solution for preventing and/or treating pathogenic infections in a subject in need thereof.

2. Related Art

Cystic fibrosis (CF) is a genetic disorder known to be an inherited disease of the secretory glands, including the glands that make mucus and sweat. Cilial epithilial cells in the CF patient have a mutated protein that instead of creating the right resin that is used to prevent the alveoli from collapsing, it makes a thicker resin, mucus. This makes the oxygen extraction in the alveoli harder as the molecules must travel through the mucus leading to breathlessness. Since the mucus stays there most of the time bacteria will thrive in it, causing multiple chest infections.

Lung disease results from clogging the airways due to mucosa build-up and resulting inflammation. Inflammation and infection cause injury and structural changes to the lungs, leading to a variety of symptoms. In the early stages, incessant coughing, copious phlegm production, and decreased ability to exercise are common. Many of these symptoms occur when bacteria that normally inhabit the thick mucus grow out of control and cause pneumonia. In later stages of CF, changes in the architecture of the lung further exacerbate chronic difficulties in breathing. In addition to typical bacterial infections, people with CF more commonly develop other types of lung disease. Among these is allergic bronchopulmonary aspergillosis, in which the body's response to the common fungus Aspergillus fumigatus causes worsening of breathing problems. Another is infection with Mycobacterium avium complex (MAC), a group of bacteria related to tuberculosis, which can cause further lung damage and does not respond to common antibiotics. Mucus in the paranasal sinuses is equally thick and may also cause blockage of the sinus passages, leading to infection. This may cause facial pain, fever, nasal drainage, and headaches. Individuals with CF may develop overgrowth of the nasal tissue (nasal polyps) due to inflammation from chronic sinus infections. These polyps can block the nasal passages and increase breathing difficulties.

Many CF patients are on one or more antibiotics at all times, even when they are considered healthy, to suppress infection(s) as much as possible. Antibiotics are absolutely necessary whenever pneumonia is suspected or there has been a noticeable decline in lung function. Antibiotics are usually chosen based on the results of a sputum analysis and the patients past response. Many bacteria common in cystic fibrosis are resistant to multiple antibiotics and require weeks of treatment with intravenous antibiotics such as vancomycin, tobramycin, meropenem, ciprofloxacin, and piperacillin. This prolonged therapy often necessitates hospitalization and insertion of a more permanent IV such as a PICC line or Port-a-Cath. Inhaled therapy with antibiotics such as tobramycin and colistin is often given for months at a time in order to improve lung function by impeding the growth of colonized bacteria. Oral antibiotics such as ciprofloxacin or azithromycin are given to help prevent infection or to control ongoing infection. Some individuals spend years between hospitalizations for antibiotics, whereas others require several antibiotic treatments each year.

### BRIEF SUMMARY OF THE INVENTION

The invention provides compositions for the treatment and prophylactic management of bacterial, mycobacterial and fungal respiratory infections in a subject in need thereof. The invention may be implemented as follows.

According to one aspect of the invention, a composition for the treatment and/or prophylactic management of respiratory infections may include about 0.01% to about 1% cetylpyridinium chloride and about 1% to about 3.4% sodium chloride, where the composition is inhaled or is an oropharyngeal gargle solution. The composition may include about 0.07% of cetylpyridinium chloride and about 3.0% of sodium chloride. The composition may include about 0.05% cetylpyridinium chloride and about 1% sodium chloride. The respiratory infections may be bacterial, mycobacterial, fungal, or viral respiratory infections.

The combination of the cetylpyridinium chloride and sodium chloride may have synergistic effects resulting in improved bacterial, mycobacterial, fungal, and viral death. The composition may be a solution or dry powder. The composition may be delivered using a jet and/or vibrating mesh nebulizer, handheld atomizer, pressurized meter dose inhaler or dry powder inhaler.

The composition may be packaged in blow-fill-seal vials, glass vials, ampoules, bottles, plastic containers or foil packs. The composition may be sterile, non-pryogenic, and preservative free. The composition may be packaged in pressurized meter dose inhalers. The composition packaged in pressurized meter dose inhalers may be sterile, non-pryogenic, and preservative free.

The dry powder may be delivered in single dose packets or multiple dose devices. The composition may be a single therapeutic. The composition may be used concomitantly, in combination therapy, or as a prophylactic agent with other anti-infective agents. The composition may have a pH of about pH 4 to about pH 8.

According to another aspect of the invention, a method of treating or preventing pathogenic infection in a subject in need thereof may include providing a composition containing about 0.01% to about 1% cetylpyridinium chloride and about 1% to about 3.4% sodium chloride to the subject in need thereof. The composition may be an oropharyngeal gargle solution. The composition may be suitable for administration by inhalation. The subject in need thereof is a subject afflicted with cystic fibrosis. The composition may include about 0.07% of cetylpyridinium chloride and about 3.0% of sodium chloride. The composition may include about 0.05% cetylpyridinium chloride and about 1% sodium chloride. The composition may be used concomitantly, in combination therapy, or as a prophylactic agent with other anti-infective agents.

Additional features, advantages, and embodiments of the invention may be set forth or apparent from consideration of the following detailed description and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is to be noted that as used herein and in the appended claims, the singular forms “a,” “an,” and “the” include the plural reference unless the context clearly dictates otherwise. This, for example, a reference to “a bacterium” is a reference to one or more bacterium and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32 etc., are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Moreover, provided immediately below is a “Definition” section, where certain terms related to the invention are defined specifically. Particular methods, devices, and materials are described, although any methods and materials similar or equivalent to those described herein can be used in the practice or testing of the invention. All references referred to herein are incorporated by reference herein in their entirety.

### DEFINITIONS

CF is cystic fibrosis

TB is Tuberculosis

PCP is Pneumocystis pneumonia

CPC is Cetylpyridinium chloride

The terms “active agent,” “drug” and “pharmacologically active agent,” as used herein, are used interchangeably to refer to a chemical material or compound which, when administered to an organism (human or animal) induces a desired pharmacologic effect. Derivatives and analogs of those compounds or classes of compounds specifically mentioned that also induce the desired pharmacologic effect are included.

The term “pharmaceutically acceptable carrier,” as used herein, refer to a material suitable for drug administration and not biologically or otherwise undesirable, i.e., that may be administered to an individual along with an active agent without causing any undesirable biological effects or interacting in a deleterious manner with any of the other components of the pharmaceutical formulation in which it is contained.

The term “pharmacologically acceptable,” as used herein, refers to a salt, ester or other derivative of an active agent provided herein that is a salt, ester or other derivative that is not biologically or otherwise undesirable.

The term “quaternary ammonium cation,” as used herein, generally refers to positively charged polyatomic ions of the structure of Formula I below:

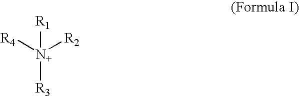

where R may be an alkyl group or hydrogen. Quaternary ammonium cations are permanently charged, independent of the pH of their solution. Quaternary ammonium cations may be synthesized by complete alkylation of ammonia or other amines.

The term “alkyl,” by itself or as part of another substituent, means, unless otherwise stated, a straight (i.e., unbranched) or branched carbon chain, or combination thereof, which may be fully saturated, mono- or polyunsaturated and can include di- and multivalent radicals, having the number of carbon atoms designated (i.e., C₁-C₁₀ means one to ten carbons). Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “quaternary ammonium salts” or “quaternary ammonium compounds,” as used herein are salts of quaternary ammonium cations with an anion. Examples of quaternary compounds include cetylpyridinium chloride, benzalkonium chloride, carnitine, benzilone, betaine, fentonium, quinapyramine, poldine, succinylmonocholine, metocurine, thioflavin, choline chloride, and other compounds known by those of skill in the art.

The term “pharmaceutically acceptable salts” is meant to include salts of the active compounds that are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, oxalic, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain specific compounds of the invention contain both basic and acidic functionalities that allow the compounds to be converted into either base or acid addition salts.

Thus, the compounds of the invention may exist as salts, such as with pharmaceutically acceptable acids. The invention includes such salts. Examples of such salts include hydrochlorides, hydrobromides, sulfates, methanesulfonates, nitrates, maleates, acetates, citrates, fumarates, tartrates (e.g., (+)-tartrates, (−)-tartrates, or mixtures thereof including racemic mixtures), succinates, benzoates, and salts with amino acids such as glutamic acid. These salts may be prepared by methods known to those skilled in the art.

The neutral forms of the compounds are preferably regenerated by contacting the salt with a base or acid and isolating the parent compound in the conventional manner. The parent form of the compound differs from the various salt forms in certain physical properties, such as solubility in polar solvents.

The terms “effective amount” or “therapeutically effective amount” of an agent as provided herein refer to a nontoxic but sufficient amount of the agent to provide the desired therapeutic effect. The exact amount required will vary from subject to subject, depending on the age, weight, and general condition of the subject, the severity of the condition being treated, the judgment of the clinician, and the like. Thus, it is not possible to specify an exact “effective amount.” However, an appropriate “effective” amount in any individual case may be determined by one of ordinary skill in the art using only routine experimentation. Those skilled in the clinical and pharmacological arts will be able to determine these factors through routine experimentation consisting of monitoring the subject and adjusting the dosage. Remington: The Science and Practice of Pharmacy (Gennaro ed. 20th edition, Williams & Wilkins Pa., USA) (2000).

The terms “treating” and “treatment” and “prophylactic” as used herein refer to reduction in severity and/or frequency of symptoms, elimination of symptoms and for underlying cause, prevention of the occurrence of symptoms and/or their underlying cause, and improvement or remediation of damage. Thus, for example, the present method of “treating” individuals with a pathogenic infection, such as TB or PCP, as the term “treating” is used herein, encompasses treatment to a clinically symptomatic individual.

The term “subject,” as used herein, includes individuals who require intervention or manipulation due to a exposure or infection with a pathogen. Furthermore, the term “subject” includes non-human animals and humans.

The term “pathogen,” or “pathogenic,” as used herein, generally refers to a biological agent that causes disease or death in a subject. Pathogens may include viruses (e.g., adenovirus, picornavirus, herpesvirus, hepadnavirus, flavivirus, retrovirus, othromyxovirus, paramyxoviris, paramyxovirus. rhabdovirus, togavirus), bacteria (e.g., mycobacterium tuberculosis, streptococcus, pseudomonas, shigella, campylobacter, salmonella), and fungal infections.

The term “anti-pathogenic” or “anti-pathogenic efficacy,” are used interchangeably, and generally refers to a substance that is capable of killing a pathogen and/or to protect a subject against pathogenic infection/growth.

“Saline,” or “saline solution,” are commonly used general terms, which refer to a sterile solution of sodium chloride in water. The solution may be used for nasal irrigation, oral administration; and inhalation forms.

As used herein, the term “saline” refers to salt, usually sodium chloride, but can include salt of potassium, magnesium or calcium. Physiological saline contains 0.9% of sodium chloride in water and is isotonic (i.e. having same osmotic pressure as blood serum).

As used herein, the term “hypertonic” refers to a solution with a solute concentration that is higher than that inside cells present in that solution, and therefore causes water to diffuse out of the cells. The term “hypertonic” is a relational term expressing the greater relative solute concentration of one solution compared with another (i.e., the latter is “hypertonic” to the former). A hypertonic solution has a lower water potential than a solution that is hypotonic to it and has a correspondingly greater osmotic pressure.

As used herein, the term “osmotic activity” refers to the net diffusion of water across a selectivity permeable membrane that is permeable in both directions to water, but varying permeable to solutes, wherein the water diffuses from one solution into another of lower water potential. Hypertonic saline solutions in concentrations greater than 1% have shown to be bactericidal and bacteristatic. In general, if an antibacterial agent is bacteriostatic, the agent essentially stops bacterial cell growth (but does not kill the bacteria); if the agent is bactericidal, the agent kills the bacterial cell (and may stop growth before killing the bacteria).

### Methods

The invention relates generally to compositions for the treatment and prophylactic management of respiratory infections. In one aspect, methods are provided for treating and/or preventing pathogenic infection in a subject. The compositions of the invention may be effective both in vitro and in vivo against pathogens, including without limitation, viruses, fungi, gram positive, gram negative bacteria and gram neutral bacteria, including, but not limited to, Staphylococcus aureus, Pseudomonas aeruginosa, Streptococcus pyogenes, Haemophilus influenzae, Listeria monocytogenes, Moraxella cattarrhalis, Salmonella typhimurium, Mycobacterium tuberculosis and E. coli and the fungi Pneumocystis jirovecii, Alternaria alternata, Aspergillus niger, Penicillium funiculosum, Fusarium solani, and Candida albicans.

In one embodiment, a method for treating pathogenic infection in a subject includes administering to a subject in need thereof a composition containing an effective amount of at least one active compound in combination with a saline solution, and specifically a hypertonic saline solution. In another embodiment, a method for preventing pathogenic infection in a subject includes administering prophylactically to a subject in need thereof a composition containing an effective amount of at least one active ingredient in combination with a saline solution, and specifically a hypertonic saline solution.

The compositions of the invention, when inhaled or used as an oropharyngeal gargle solution or rinse, have direct cellular contact without having to be ingested, injected or metabolized. The inhaled modality or oropharyngeal gargle solution or rinse delivers the active agents directly to the respiratory system (e.g., lungs, pharynx, sinus, trachea, bronchi, oral cavity, and so on) where the bacterial and fungal pathogens are harbored in pulmonary mucus. Additionally, since the compositions disclosed herein have a separate delivery pathway and anti-pathogenic activity than that of systemically delivered agents, the composition may be used concomitantly or in combination therapy with other treatments.

Compositions are provided herein that are suitable for such methods. The compositions include at least one active ingredient that has anti-pathogenic efficacy in a saline solution that is suitable for oral administration and/or inhalation. In a specific embodiment, the at least one active ingredient is a cationic quaternary ammonium compound, and in a more specific embodiment, the cationic quaternary ammonium compound is cetylpyridinium chloride (CPC). CPC has the molecular formula C₂₁H₃₈NCl and has the structure of Formula II below:

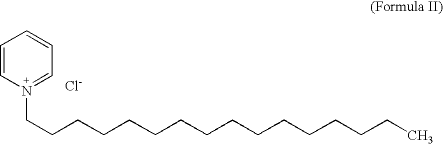

In some embodiments, the composition may contain CPC in combination with sodium chloride where the sodium chloride is present in an amount to render the composition hypertonic. In particular, the CPC may be present in the composition in a range of about 0.01% to about 1.0% and sodium chloride may be present in the composition in a range of about 1% to about 3.4%. In one specific embodiment, the composition may contain about 0.07% of CPC and about 3.0% of sodium chloride. In another specific embodiment, the composition may contain about 0.05% CPC and about 1.0% sodium chloride.

According to other embodiments, the composition may be a CPC/hypertonic saline solution having a pH in the range of about pH 4 to about pH 8, and in particular from about pH 5 to about pH 7.5. The pH may be adjusted by the addition of a solution containing an acidic salt or an acid (e.g., hydrochloric or sulfuric acid); or of the basic salt or a base (e.g., sodium hydroxide). In case of insufficient stability of the formulation, aqueous buffered systems (e.g., citrate, acetate, phosphate) may be added to keep the pH with a physiologically acceptable range. A variety of buffers known in the art may be used in the compositions of the invention, non-limiting examples include various salts of organic or inorganic acids, bases, or amino acids, and including various forms of citrate, phosphate, tartrate, succinate, adipate, maleate, lactate, acetate, bicarbonate, or carbonate ions.

### Synergistic Effect

While not intending to be limited to a particular mechanism, in some embodiments, the mechanism action of CPC occurs by binding to the cell membrane thereby disrupting the osmotic integrity of the cellular membrane. Disruption of the membrane causes leakage of intracellular potassium, magnesium, sugars, and metabolites resulting in cellular death. Additionally, CPC functions as a virucidal by disrupting the viral capsid of both enveloped and non-enveloped viruses. Hypertonic saline solutions function by creating mucocillary mobilization or motility of bacterial and fungal pathogens that harbor in pulmonary mucus. The solute concentration causes anti-pathogenic activity by diffusing water out of the cells. The osmotic property creates mucocillary mobilization by the thinning of the mucus or liquidization or lowering the viscosity of the mucus allowing greater surface contact of agents to microorganisms.

It is not uncommon for the effect of two chemicals on an organism to be greater than the effect of each chemical individually, or the sum of the individual effects. A “synergistic effect” refers to the presence of one chemical enhancing the effects of the second chemical. Until the compositions of the invention were discovered by the inventors, the synergistic anti-pathogenic activity resulting from a composition containing a combination of a cationic quaternary ammonium compounds, such as CPC and hypertonic saline was not appreciated. The synergistic effect of the composition of the invention exposes more pathogens to the combination-agent and provides a greater and quicker “kill” effect of the cells and/or virus than if used a single or separate agents alone.

The combined synergistic effects of the combination of CPC and hypertonic saline in the compositions of the invention are three fold. First, the hypertonic sodium chloride osmotically lowers the viscosity of the mucus allowing greater surface contact of the CPC to the pathogens where the cationic effect of the CPC disrupts osmotic integrity of the cellular membranes. The anti-pathogenic properties of both CPC and hypertonic sodium chloride are synergistically enhanced with the osmotically of the hypertonic of the sodium chloride to draw-out cellular metabolites of the microorganisms cell membranes that have been disrupted or breached by the cationic effects of the CPC. Additionally, the virucidal properties of both CPC and hypertonic sodium chloride individually are synergistically enhanced with the osmotically of the hypertonic of the sodium chloride.

### Formulations

The compositions of the invention may be a solution or dry powder. In one embodiment, the composition is a solution that may be delivered using a jet and/or vibrating mesh nebulizer, handheld atomizer, pressurized meter dose inhaler or dry powder inhaler. In another embodiment, the composition is a dry powder that may be delivered in single dose packets or multiple dose devices.

In another embodiment, the invention provides pharmaceutical compositions comprising CPC in combination with a pharmaceutically acceptable excipient (e.g., carrier). Suitable pharmaceutically acceptable carriers include water, salt solutions (such as Ringer's solution), alcohols, oils, gelatins, and carbohydrates such as lactose, amylose or starch, fatty acid esters, hydroxymethycellulose, and polyvinyl pyrrolidine. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention.

The compositions of the invention can be administered alone or can be co-administered to the subject. Co-administration is meant to include simultaneous or sequential administration of the compounds individually or in combination (more than one compound). The preparations can also be combined, when desired, with other active substances (e.g. antibiotics). For example, the compounds of the invention may be co-administered with mucolytics, antihistamines, antibiotics, antifungals, and the like.

The CPC compositions of the invention may be prepared and administered in a wide variety of oral, parenteral, and topical dosage forms. Also, the compositions described herein can be administered by inhalation, for example, intranasally. Accordingly, the invention also provides CPC compositions comprising a pharmaceutically acceptable carrier or excipient.

Liquid form preparations include solutions, suspensions, and emulsions, for example, water or water/propylene glycol solutions. Aqueous solutions suitable for oral use can be prepared by dissolving the active component in water and adding suitable colorants, flavors, stabilizers, and thickening agents as desired. Flavoring agents which are used in the practice of the present invention include essential oils as well as various flavoring aldehydes, esters, alcohols, and similar materials. Examples of the essential oils include oils of spearmint, peppermint, wintergreen, sassafras, clove, sage, eucalyptus, marjoram, cinnamon, lemon, lime, grapefruit, and orange. Also useful are such chemicals as menthol, carvone, and anethole. The flavoring agent is incorporated in the oral composition at a concentration of about 0.1 to about 5% by weight and preferably about 0.5 to about 1.5% by weight. Various other materials may be incorporated oral compositions of this invention, including desensitizers, such as potassium nitrate; whitening agents, such as hydrogen peroxide, calcium peroxide and urea peroxide; preservatives; silicones; and chlorophyll compounds. These additives, when present, are incorporated in the oral compositions of the present invention in amounts which do not substantially adversely affect the properties and characteristics desired.

Aqueous suspensions suitable for oral use can be made by dispersing the finely divided active component in water with viscous material, such as natural or synthetic gums, resins, methylcellulose, sodium carboxymethylcellulose, and other well-known suspending agents.

Also included are solid form preparations that are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents, and the like.

### Dosages

The pharmaceutical preparation is preferably in unit dosage form. In such form the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of active component in a unit dose preparation may be varied or adjusted from 0.1 mg to 10000 mg, more typically 1.0 mg to 1000 mg, most typically 10 mg to 500 mg, according to the particular application and the potency of the active component. The composition can, if desired, also contain other compatible therapeutic agents.

Some compounds may have limited solubility in water and therefore may require a surfactant or other appropriate co-solvent in the composition. Such co-solvents include: Polysorbate 20, 60, and 80; Pluronic F-68, F-84, and P-103; cyclodextrin; and polyoxyl 35 castor oil. Such co-solvents are typically employed at a level between about 0.01% and about 2% by weight.

Viscosity greater than that of simple aqueous solutions may be desirable to decrease variability in dispensing the formulations, to decrease physical separation of components of a suspension or emulsion of formulation, and/or otherwise to improve the formulation. Such viscosity building agents include, for example, polyvinyl alcohol, polyvinyl pyrrolidone, methyl cellulose, hydroxy propyl methylcellulose, hydroxyethyl cellulose, carboxymethyl cellulose, hydroxy propyl cellulose, chondroitin sulfate and salts thereof, hyaluronic acid and salts thereof, and combinations of the foregoing. Such agents are typically employed at a level between about 0.01% and about 2% by weight.

The compositions of the invention may additionally include components to provide sustained release and/or comfort. Such components include high molecular weight, anionic mucomimetic polymers, gelling polysaccharides, and finely-divided drug carrier substrates. These components are discussed in greater detail in U.S. Pat. Nos. 4,911,920; 5,403,841; 5,212,162; and 4,861,760. The entire contents of these patents are incorporated herein by reference in their entirety for all purposes.

The dosage of the composition of the invention to be administered can be determined without undue experimentation and will be dependent upon various factors including the nature of the active agent (whether metal bound or metal free), the route of administration, the subject, and the result sought to be achieved. A suitable dosage of the compound to be administered orally can be expected to be in the range of about 0.01 to about 50 mg/kg/day, and more particularly, in the range of about 0.1 mg/kg/day to about 10 mg/kg/day. For aerosol administration, it is expected that the dose will be in the range of about 0.001 mg/kg/day to about 5/. Mg/kg/day, and more specifically, in the range of about 0.01 mg/kg/day to about 1 mg/kg/day. Suitable doses of the compounds will vary, for example, with the compound and with the result sought.

In certain embodiments, the compositions of the invention may be administered prophylactically to serve as a protectant against exposure to pathogenic infection. For any composition described herein, the therapeutically effective amount can be initially determined from cell culture assays. Target concentrations will be those concentrations of active compound(s) that are capable of preventing and/or treating pathogenic infection. Methods for assessing the anti-pathogenic efficacy of the composition is known by those of skill in the art and is matter of routine experimentation.

Therapeutically effective amounts for use in humans may be determined from animal models. For example, a dose for humans can be formulated to achieve a concentration that has been found to be effective in animals. The dosage in humans can be adjusted by monitoring the kill rate of pathogenic infection and adjusting the dosage upwards or downwards.

Dosages may be varied depending upon the requirements of the patient and the compound being employed. The dose administered to a patient, in the context of the invention, should be sufficient to effect a beneficial therapeutic response in the patient over time. The size of the dose also will be determined by the existence, nature, and extent of any adverse side effects. Generally, treatment is initiated with smaller dosages, which are less than the optimum dose of the compound. Thereafter, the dosage is increased by small increments until the optimum effect under circumstances is reached.

Dosage amounts and intervals can be adjusted individually to provide levels of the administered composition effective for the particular indication being treated. This will provide a therapeutic regimen that is commensurate with the severity of the individual's pathogenic infection.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of active compound by considering factors such as compound potency, relative bioavailability, patient body weight, presence and severity of adverse side effects, preferred mode of administration, and the toxicity profile of the selected agent.

The composition may be packaged in several ways, including but not limited to, blow-fill-seal vials, glass vials, ampoules, bottles, plastic containers and foil packs. In one embodiment, the composition may be packaged in pressurized meter dose inhalers. Additionally, the composition may be sterile, non-pryogenic, and preservative free.

Without further elaboration, it is believed that one skilled in the art using the preceding description can utilize the invention to the fullest extent. The following examples are illustrative only, and not limiting of the disclosure in any way whatsoever.

### EXAMPLES

### Specific Example 1

To confirm the activity of Pseudomonas found in CF patients, a standard time-kill test was conducted that compared the biocidal efficacy of composition containing 0.05% CPC and about 1.0% sodium chloride on a sample of P. aeruginose with CF sputum and a sample of P. aeruginose without CF sputum. The results of the time kill test show that the sample without CF sputum that came into contact with the composition had significantly decreased bacterial colony counts to undetectable counts by 1 hour. This was equivalent to about a 5-log decrease. In the presence of CF sputum, at 1 hour the log decrease was about 1 log, representing about an 80.4% CFU reduction. At 24 hours, a 1½ log killing was seen, which was equivalent to about a 97.5% CFU reduction.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in cellular and molecular biology, chemistry, or in the relevant fields are intended to be within the scope of the appended claims.

The disclosures of all references and publications cited above are expressly incorporated by reference in their entireties to the same extent as if each were incorporated by reference individually.

What is claimed is:

1. A composition for treatment and prophylactic management of respiratory infections, the composition comprising:
about 0.01% to about 1% cetylpyridinium chloride;
about 1% to about 3.4% sodium chloride; and
wherein the composition is inhaled or is an oropharyngeal gargle solution.

2. The composition of claim 1, wherein the composition comprises about 0.07% of cetylpyridinium chloride and about 3.0% of sodium chloride.

3. The composition of claim 1, wherein the composition comprises about 0.05% cetylpyridinium chloride and about 1% sodium chloride.

4. The composition of claim 1, wherein the respiratory infections are bacterial, mycobacterial, fungal, or viral respiratory infections.

5. The composition of claim 1, wherein a combination of the cetylpyridinium chloride and the sodium chloride has synergistic effects resulting in improved bacterial, mycobacterial, fungal, and viral death.

6. The composition of claim 1, wherein the composition is a solution or dry powder.

7. The composition of claim 6, wherein the composition is delivered using a jet and/or vibrating mesh nebulizer, handheld atomizer, pressurized meter dose inhaler or dry powder inhaler.

8. The composition of claim 1, wherein the composition is packaged in blow-fill-seal vials, glass vials, ampoules, bottles, plastic containers or foil packs.

9. The composition according to claim 1, wherein the composition is sterile, non-pryogenic, and preservative free.

10. The composition of claim 7, wherein the composition is packaged in pressurized meter dose inhalers.

11. The composition of claim 10, wherein the composition packaged in pressurized meter dose inhalers is sterile, non-pryogenic, and preservative free.

12. The composition of claim 6, wherein the dry powder is delivered in single dose packets or multiple dose devices.

13. The composition of claim 1, wherein the composition is a single therapeutic.

14. The composition of claim 1, wherein the composition is used concomitantly, in combination therapy, or as a prophylactic agent with other anti-infective agents.

15. The composition of claim 1, wherein the compositions has a pH of about pH 4 to about pH 8.

16. A method of treating or preventing pathogenic respiratory infection in a subject in need thereof, said method comprising the step of:
providing a composition containing about 0.01% to about 1% cetylpyridinium chloride and about 1% to about 3.4% sodium chloride to the subject in need thereof.

17. The method of claim 16, wherein the composition is an oropharyngeal gargle solution.

18. The method of claim 16, wherein the composition is suitable for inhalation.

19. The method of claim 16, wherein the subject in need thereof is a subject afflicted with cystic fibrosis.

20. The composition of claim 16, wherein the composition comprises about 0.07% of cetylpyridinium chloride and about 3.0% of sodium chloride.

21. The composition of claim 16, wherein the composition comprises about 0.05% cetylpyridinium chloride and about 1% sodium chloride.

22. The composition of claim 16, wherein the composition is used concomitantly, in combination therapy, or as a prophylactic agent with other anti-infective agents.
